(19) United States
(12) Patent Application Publication
(10) Pub. No.: US 2012/0143015 A1
(43) Pub. Date: Jun. 7, 2012

(54) DIAGNOSTIC METHOD FOR ANALYZING, REMEDIATING AND MAINTAINING THE HOMEOSTATIC RELATIONSHIP OF SUBSTANCES IN AN INDIVIDUAL'S BODY

(76) Inventor: William J. McFaul, Jackson, NJ (US)

(21) Appl. No.: 13/310,212

(22) Filed: Dec. 2, 2011

Related U.S. Application Data
(60) Provisional application No. 61/419,058, filed on Dec. 2, 2010.

(51) Int. Cl. A61B 5/00

(52) U.S. Cl. 600/300

(57) ABSTRACT

A diagnostic method and system for determining the relationship between various substances that constitute or affect the formation of RNA molecules. These substances affect the production of RNA, which activates and maintains interactions between other bodily substances that include but are not limited to, proteins, hormones, peptides, enzymes, amino acids, bacteria, viruses, fungi, etc. in the body. The diagnostic method provides the appropriate information to a physician to identify an appropriate treatment to maintain or achieve a homeostatic relationship between the aforementioned substances that comprise or affect the formation of RNA molecules to enable the adequate production of RNA molecules to effectively manage interactions between other bodily substances. A homeostatic relationship between the substances that comprise or affect the formation of RNA therefore enables the establishment of proper interrelationships and interactions between other bodily substances regulated by RNA so as to ensure wellness and prevent chronic diseases.

### CROSS-REFERENCE TO RELATED APPLICATIONS

This application is a non-provisional of and claims the benefit of priority to U.S. Provisional Patent Application No. 61/419,058 on filed Dec. 2, 2010, the entire disclosure of which is herein incorporated by reference in its entirety.

### BACKGROUND OF THE INVENTION

1. Field of the Invention

The present invention relates to the field of diagnostic methods for analyzing and maintaining or correcting the levels of substances in the body of individuals based on diagnostic assessments of those substance levels. In particular, the present invention is directed to a process for assessing substances that comprise and/or affect the formation of RNA molecules in order to determine an appropriate therapeutic treatment to maintain the adequate interactions between those substances in the body.

2. Description of the Related Technology

All forms of life are supported by a variety of substances, including but not limited to hormones, proteins, peptides, fatty acids, cells, bacteria, viruses and fungi. These substances must remain in the state of homeostasis to maintain life for humans as well as plants and animals.

Current scientific findings do not recognize that various pairs of substances, specifically pairs of substances that comprise or affect the formation of RNA, in the body affect one another. Furthermore, it is not recognized that modulators exist that regulate these pairs of substances. It has further not been appreciated that certain substances are a part of more than one modulated pair of substances. It is an objective of the present invention to utilize the relationships between these pairs of substances and their respective modulators to determine and assess various physiological parameters affecting the health of an individual.

Therefore, there is a need to utilize the relationships between pairs of biological substances and their respective modulators to determine and assess various physiological parameters of an individual in order to assess health and determine an appropriate therapeutic treatment to establish homeostasis of the pairs of substances that comprise or affect the formation of RNA. Specifically, there is a need to identify and utilize the homeostatic relationship between the substances that comprise or affect the formation of RNA molecules in order to assess and ensure that they are present in a sufficient amount, concentration and level to maintain efficient interactions for establishing a healthy, homeostatic biological environment.

### SUMMARY OF THE INVENTION

An object of the invention is establishing methods to assess the levels of substances necessary to ensure homeostasis and to add or subtract minerals or amino acids to sustain homeostasis (balance) of RNA molecules or remediate levels of substances that affect the formation of or constitute RNA molecules if disruptions (imbalances) occur.

An aspect of the present invention may be a method for establishing a diagnosis based upon compared levels of substances that affect the formation of or comprise mRNA IL-4 comprising: measuring a level of nickel in an individual; measuring a level of zinc in the individual; measuring a level of boron in the individual; comparing the measured levels; establishing a diagnosis based upon the compared measured levels; adding or subtracting an additional amount of nickel in the individual to ensure homeostasis based upon the compared measured levels and the established diagnosis; adding or subtracting an additional amount of zinc in the individual to ensure homeostasis based upon the compared measured levels and the established diagnosis; adding or subtracting an additional amount of boron in the individual to ensure homeostasis based upon the compared measured levels and the established diagnosis.

An aspect of the present invention may be a method for establishing a diagnosis based upon compared levels of substances affecting the formation of or comprising mRNA IL-6 comprising: measuring a level of calcium in the individual; measuring a level of magnesium in the individual; measuring a level of chloride in the individual; comparing the measured levels; establishing a diagnosis based upon the compared measured levels; adding or subtracting an additional amount of the mineral calcium in the individual to ensure homeostasis based upon the compared measured levels and the established diagnosis; adding or subtracting an additional amount of the mineral magnesium in the individual to ensure homeostasis based upon the compared measured levels and the established diagnosis; and adding or subtracting an additional amount of the chloride in the individual to ensure homeostasis based upon the compared measured levels and the established diagnosis.

An aspect of the present invention may be a method for establishing a diagnosis based upon compared levels of substances affecting the formation of or comprising mRNA IL-1 comprising: measuring a level of aluminum in the individual; measuring a level of the iron in the individual; measuring a level of the phosphorous in the individual; comparing the measured levels; establishing a diagnosis based upon the compared measured levels; adding or subtracting an additional amount of aluminum in the individual based upon the compared measured levels and the established diagnosis; adding or subtracting an additional amount of iron in the individual based upon the compared measured levels and the established diagnosis; and adding or subtracting an additional amount of phosphorous in the individual based upon the compared measured levels and the established diagnosis.

These and various other advantages and features of novelty that characterize the invention are pointed out with particularity in the claims annexed hereto and forming a part hereof. However, for a better understanding of the invention, its advantages, and the objects obtained by its use, reference should be made to the drawings which form a further part hereof, and to the accompanying descriptive matter, in which there is illustrated and described a preferred embodiment of the invention.

### BRIEF DESCRIPTION OF THE DRAWINGS

FIG. 1 illustrates the relationship of a modulated pair.

FIG. 2 shows the relationship between nickel, zinc and boron (which comprise or affect the substances forming mRNA IL-4).

FIG. 3 shows the relationship between calcium, magnesium and chloride (which comprise or affect the substances forming mRNA IL-6).

FIG. 4 is a flow chart showing the method for establishing a diagnosis.

FIG. 5 shows the relationship between aluminum, iron and phosphorous (which comprise or affect the substances forming mRNA IL-1).

FIG. 6 is a flow chart showing the method for establishing a diagnosis.

### DETAILED DESCRIPTION OF THE PREFERRED EMBODIMENT(S)

All forms of life are supported by modulated pairs of substances, including but not limited to, hormones, proteins, peptides, fatty acids, viruses, bacteria and fungi. These substances create the homeostasis essential to maintain life for humans as well as plants and animals. Interactions and corollary relationships between these substances in the body are activated and managed by RNA molecules, which are similarly dependent upon the homeostatic relationship between pairs of substances affecting the formation of RNA molecules. Maintaining homeostatic relationships between the modulated pairs of substances that comprise or affect these RNA molecules is therefore important to maintaining adequate interactions between the substances in the body.

Several terms are used herein and are defined as follows:

The term “modulator” means an agent, substance or mechanism that facilitates the maintenance of homeostasis between pairs.

“Pairs” may be minerals, proteins, peptides, hormones, enzymes, amino acids, substances that form or comprise RNA molecules, bacteria, virus, or fungus. The agent can decrease intensity or levels of other substances, or increase intensity or levels of compensatory ones. In terms of physics and chemistry, modulators would be referred to as neutron and transitional elements respectively.

The term “catalyst” means the driver which initiates modulation to take place. Such catalysts can include enzymes, hormones, RNA, and countless signaling mechanisms.

The term “Homeostatic Relationship (Homeostasis)” means the balance or equilibrium between two substances. The relationship does not have to be 50%-50% but can vary depending upon the two substances in question.

The term RNA is intended to include all types of ribonucleic acids, including but not limited to messenger (mRNA) and non-coding (ncRNA), such as transfer RNA (tRNA), ribosomal RNA (rRNA) and ncRNA involved in gene regulation or processing.

The invention addresses the diagnostic analysis of the corollary relationships between substances that provide and maintain homeostasis; i.e. pairs which include substances that are stimulatory/excitatory/positively charged and those that are compensatory/inhibitory/negatively charged as well as substances that perform modulatory functions to support or maintain the homeostatic relationship between substances. In particular, the invention is directed to a diagnostic method for analyzing the relationship between pairs of substances that comprise or affect the formation of RNA molecules in order to provide the necessary information for determining an appropriate therapeutic treatment to maintain or achieve a homeostatic relationship between a pair of substances that comprise or affect the formation of RNA so as to enable the production of an adequate level of RNA to achieve or maintain a medically assessed healthy state.

The data for measuring homeostasis between substances within a modulated pair can vary from one individual or specimen to another due to environmental factors, basic metabolism, genetic make-up and other factors. Accordingly, a range of homeostatic relationships will need to be determined for all modulated pairs in order to assess deviations and disruptions.

By utilizing the analysis of data relative to the ranges of homeostasis and the deviations thereof, medical science (for humans as well as well as in veterinary medicine for animals) will be able to manufacture new pharmaceutical products and other substances, manufacture point of care testing kits, prepare diets, manufacture devices, create therapies, compound personalized nutrients or substances of various types in order to maintain or regulate homeostasis.

Data derived from the application of the invention will allow for diagnostic analysis and control of levels of substances in order to prevent their depletion, to maintain adequate levels to prevent disruption as well as to restore imbalances to prevent illnesses, treat patients, eliminate the causes of disease states or develop cures.

Since nutrients, such as minerals, electrolytes, enzymes, amino acids, proteins, hormones, peptides, vitamers (vitamins), bacteria, virus, fungus, etc., are essential to create defenses for the organs, glands and operating systems of the body, as well as to provide the energy to sustain life, establishing, maintaining or restoring corollary relationships between substances that constitute “nutrients” will enable cross-category analysis to prevent depletion of the body's defense in order to prevent illness as well as to increase levels of nutritional energy to strengthen the body to offset the effects of existing ailments and diseases.

Data derived though the application of the invention will enable scientists to adjust an individual's body to compensate for the impacts of various chemicals and natural products, including herbs and foods and medications, as they can impact hormones, proteins, peptides, enzymes, the body's natural flora of bacteria, virus and fungus as well as chromosomes, RNA, genes/DNA, rates of cellular absorption and the firing rates of neurons/signaling mechanisms. An example of such impacts on the body is found in scientific studies that identify the ability of numerous chemicals to disrupt hormones and/or genes

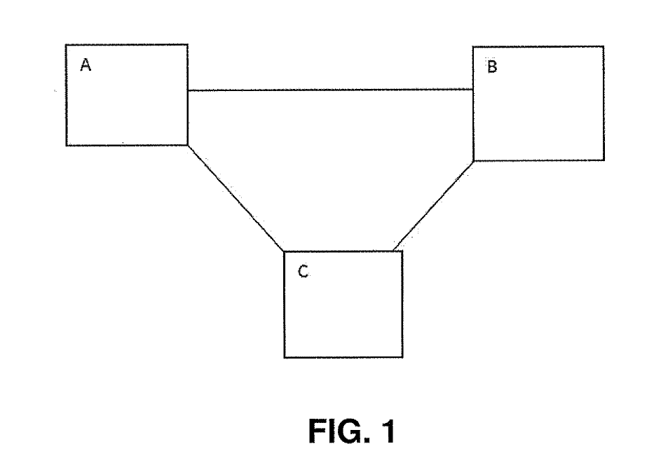

The concept of modulation in relation to homeostatic pairs is illustrated in FIG. 1. This example illustrates a relationship between two substances, A and B, and the existence of a third substance C that serves to support A or B in order to attempt to maintain an adequate level of homeostasis (balance) between the two substances.

One example of modulated pairs of substances that comprise or affect the formation of RNA in the body is calcium and magnesium modulated by chloride, which comprise or affect the substances forming mRNA IL-6. Other examples include, aluminum and iron modulated by phosphorous, which comprise or affect the substances forming mRNA IL-1, and bromine and iodine modulated by chloride, which comprise or affect the substances forming mRNA IL-10. Examples of other types of modulated pairs in the body that are not necessarily related to RNA formulation include: norepinephrine and prolactin modulated by dopamine; glucose and insulin modulated by glucagon; red blood cells and white blood cells modulated by platelets; phosphatidylcholine (PC) and phosphatidylserine (PS) modulated by dimethylaminoethanol (DMAE).

Correlation between base values expressed when the range of homeostasis is determined and individual test results can be used as a means of assessing levels or disruptions between pairs or the existence of increases in the levels of modulators in order to maintain homeostasis. Methods of diagnostic analysis may also detect the presence of catalysts as markers for the existence of a disruption within a modulated pair that the body is in the process of correcting. Detection of such catalysts may be included as a factor in the process of analyzing the correlation of substances within modulated pairs.

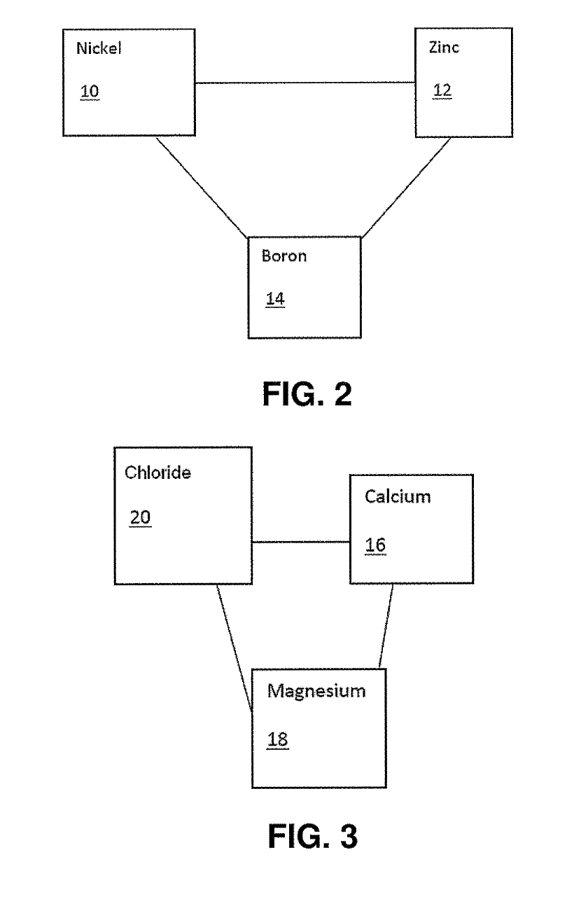

Described herein is an example of the determination of homeostatic levels of two modulated pairs discussed above. FIG. 2 shows a first pair of nickel 10 and zinc 12 modulated by boron 14 (which comprise or affect the substances forming mRNA IL-4). FIG. 3 shows a second pair of calcium 16 and magnesium 18 modulated by chloride 20 (which comprise or affect the substances forming mRNA IL-6).

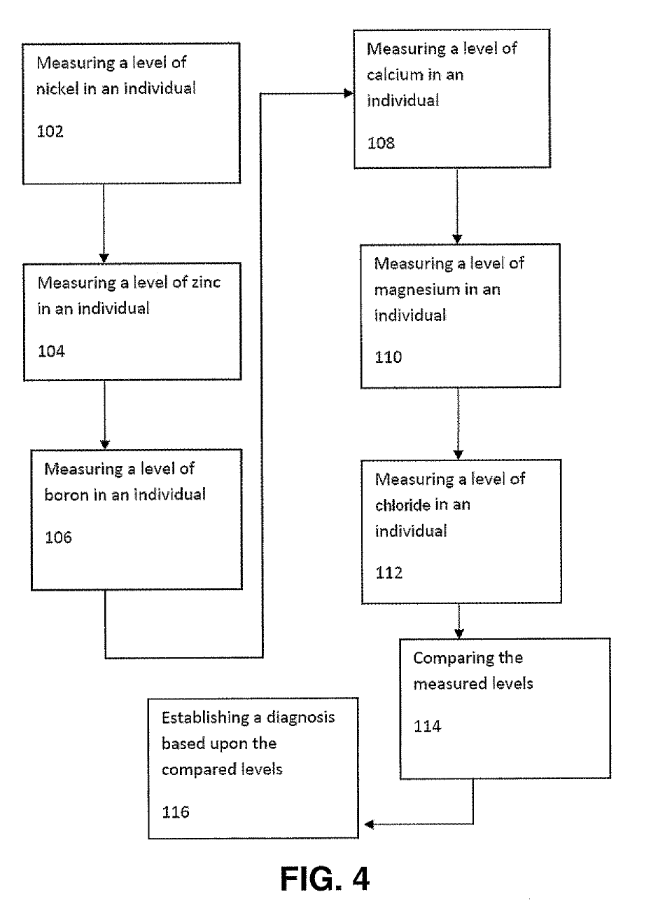

FIG. 4 shows the method of evaluating the homeostatic levels of two sets of modulated pairs in order to provide a base index for administering a substance to maintain or restore homeostasis. In step 102 the level of nickel 10 is measured in an individual. The level of nickel 10 may be measured by a variety of diagnostic tests that include but are not limited to the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat tears; saliva; mucus; phlegm; hair; fingernails; bone marrow. The measured level of nickel 10 in the body is expressed in various units depending upon the test employed in order to measure the level of the nickel 10. This level can be expressed by the variable X. There is a preferred range in which X may fall that varies depending upon the type of test that is used in order to measure the level of nickel 10. In an embodiment of the present invention a standard type of test is used.

In step 104 the level of zinc 12 in an individual is measured. This level can be measured using one of the various processes listed above. The measured level of zinc 12 is preferably expressed in the same units of measurement as that used in the measurement of nickel 10 so as to more readily compare the levels with respect to each other in order to obtain a base level. This level can be expressed by the variable Y. There is a preferred range in which Y may fall that varies depending upon the type of test that is used in order to measure the level of zinc 12. In an embodiment of the present invention a standard type of test is used.

In step 106 the level of boron 14 in an individual is measured. This level can be measured using one of the various processes listed above. The measured level of boron 14 is preferably expressed in the same units of measurement as that used in the measurement of nickel 10 so as to more readily compare the levels with respect to each other in order to obtain a base level. This level can be expressed by the variable Z. There is a preferred range in which Z may fall that varies depending upon the type of test that is used in order to measure the level of boron 14. In an embodiment of the present invention a standard type of test is used.

In step 108, the level of calcium 16 in an individual is measured. This level can be measured using one of the various processes listed above. The measured level of calcium 16 is preferably expressed in the same units of measurement as that used in the measurement of nickel 10 so as to more readily compare the levels with respect to each other in order to obtain a base level. This level can be expressed by the variable A. There is a preferred range in which A may fall that varies depending upon the type of test that is used in order to measure the level of calcium 16. In an embodiment of the present invention a standard type of test is used.

In step 110, the level of magnesium 18 in an individual is measured. This level can be measured using one of the various processes listed above. The measured level of magnesium 18 is preferably expressed in the same units of measurement as that used in the measurement of nickel 10 so as to more readily compare the levels with respect to each other in order to obtain a base level. This level can be expressed by the variable B. There is a preferred range in which B may fall that varies depending upon the type of test that is used in order to measure the level of magnesium 18. In an embodiment of the present invention a standard type of test is used.

In step 112, the level of chloride 20 in an individual is measured. This level can be measured using one of the various processes listed above. The measured level of chloride 20 is preferably expressed in the same units of measurement as that used in the measurement of nickel 10 so as to more readily compare the levels with respect to each other in order to obtain a base level. This level can be expressed by the variable C. There is a preferred range in which C may fall that varies depending upon the type of test that is used in order to measure the level of chloride 20. In an embodiment of the present invention a standard type of test is used.

In step 114, the measured levels X, Y, Z, A, B and C are then compared to standard levels. This may be done in a number of different ways. One possible way in which this can be accomplished is to total the values of all of the levels (X+Y+Z+A+B+C) in order to establish sum level N. This sum level N can be used in step 116 in order to establish a diagnosis of the individual that can be used in determining the provision of substances to add or subtract substances to maintain or restore the homeostatic relationship between targeted substances. Specifically, sum level N can be used to assess whether the substances of each pair are present in a homeostatic relationship relative to one another (e.g. whether X and Y and/or A and B establish a homeostatic relationship to enable the production of an adequate level of RNA, specifically mRNA IL-4 and mRNA IL-6, to achieve or maintain a medically assessed healthy state). This in turn may be used to determine whether the addition or remediation of one of the pairs of substances is necessary to maintain or restore the homeostatic relationship, either by adding an amount of one or more of the substances or by adding an amount of one or more modulators.

In one embodiment, the measured levels of the pairs of substances and/or their respective modulators may also be compared to standard levels. For example, N may be a summation of the total measured levels of (X+Y); (X+Y+Z); (A+B) or (A+B+C). Other methods for determining the relationship between the pair of substances, such as vector analysis and the calculation of the ratio between the levels of the substances in a pair, may also be used.

In one embodiment, the comparative standard levels of X, Y, Z, A, B, C and N may be a predetermined amount applicable to groups individuals sharing one or more characteristics (e.g. age, sex, diseases, etc.). Alternatively, the comparative standard levels of X, Y, Z, A, B, C and N may be specific to an individual medically assessed as healthy.

An example of the physiological consequences of an imbalance between the constituents of mRNA IL-4 would be when excessive nickel lowers intracellular zinc thus creating atopic dermatitis on the skin. In the body's interstitial fluid, these same imbalances would create an atopic dermatitis-like inflammation on the epithelial cells that line the bronchioles. The outcome would be asthma.

An example of the physiological consequences of an imbalance between the levels of calcium and magnesium would be excessive apoptosis (cell death). Research has linked calcium-magnesium imbalances to the cause of colorectal cancer. Prior to this invention, testing of the levels of the constituents of mRNA IL-6 to determine their homeostatic (corollary) relationships and the cause of excessive cell death has not existed.

An example of this is provided below using hypothetical numbers so as to make understanding of the process easier.

Measuring nickel 10 via a saliva test results in a number for X that results in a number 4, from within a range of 1-10. Measuring zinc 12 via a saliva test results in a number for Y of 5 from within a range of 1-10. Measuring boron 14 via a saliva test results in a number for Z of 6 from within a range of between 1-10. Measuring calcium 16 via a saliva test results in a number for A of 4 from within a range of 1-10. Measuring magnesium 18 via a saliva test results in a number for B of 6 from within a range of 1-10. Measuring chloride 20 via a saliva test results in a number for C of 1 from within a range of 1-10. These numbers are then totaled and results in an N value of 26. A value for N between 20 and 30 may indicate a normal level.

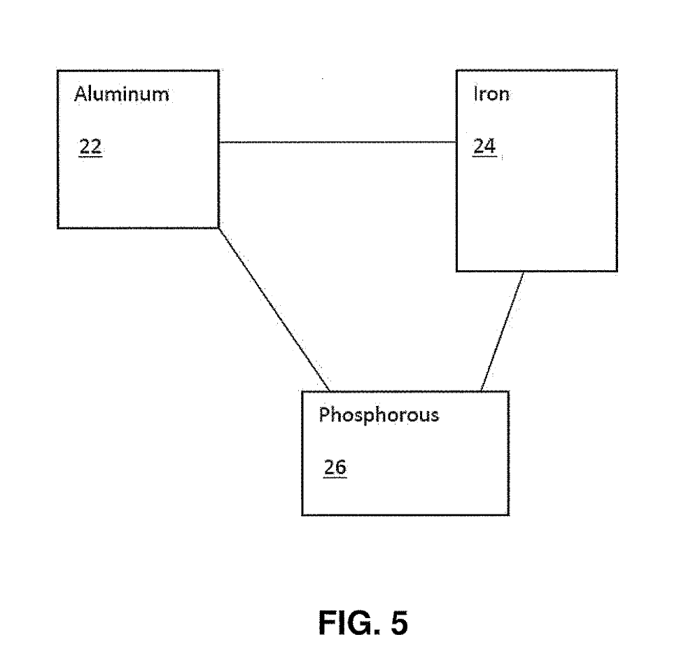

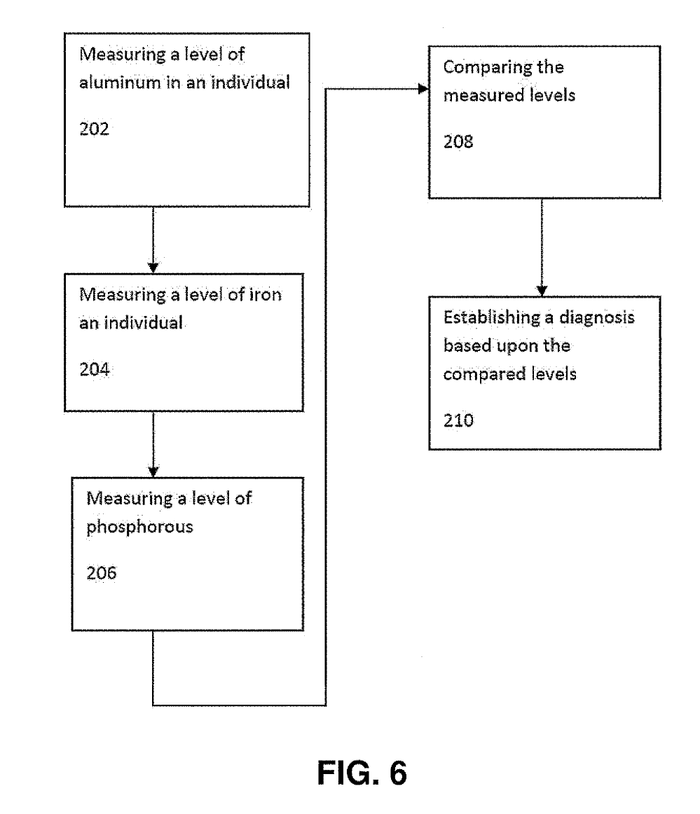

In a second set is the pair of aluminum 22 and iron 24 modulated by phosphorous 26 (which comprise or affect the production of mRNA IL-1). This relationship is shown in FIG. 5. PIG. 6 shows the method of evaluating the homeostatic levels of the modulated pair in order to provide base index for administering additional substances. In step 202 the level of aluminum 22 is measured in an individual. The level of iron 24 may be measured by a variety of diagnostic tests that include but are not limited to the analysis of diagnostic testing or imaging that indicates activity and the analysis of various fluids and excretions within and produced by the body. Examples of these fluids and excretions include: blood and its components; urine; fecal matter; collagen; chyle; interstitial fluid (tissue fluid); lymph; extracellular fluid; amniotic fluid; sweat tears; saliva; mucus; phlegm; hair; fingernails; bone marrow. The measured level of aluminum 22 in the body is expressed in various units depending upon the test employed in order to measure the level of iron 24. This level can be expressed by the variable D. There is a preferred range in which D may fall that varies depending upon the type of test that is used in order to measure the level of aluminum 22. In an embodiment of the present invention a standard type of test is used.

In step 204 the level of iron 24 in an individual is measured. This level can be measured using one of the various processes listed above. The measured level of iron 24 is preferably expressed in the same units of measurement as that used in the measurement of aluminum 22 so as to more readily compare the levels with respect to each other in order to obtain a base level. This level can be expressed by the variable E. There is a preferred range in which E may fall that varies depending upon the type of test that is used in order to measure the level of iron 24. In an embodiment of the present invention a standard type of test is used.

In step 206 the level of phosphorous 26 in an individual is measured. This level can be measured using one of the various processes listed above. The measured level of phosphorous 26 is preferably expressed in the same units of measurement as that used in the measurement of aluminum 22 so as to more readily compare the levels with respect to each other in order to obtain a base level. This level can be expressed by the variable F. There is a preferred range in which F may fall that varies depending upon the type of test that is used in order to measure the level of phosphorous 26. In an embodiment of the present invention a standard type of test is used.

In step 208, the measured levels D, E, and F are then compared to standard levels. This may be done in a number of different ways. One possible way in which this can be accomplished is to total the values of all of the levels (D+E+F) in order to establish sum level G. This sum level G can be used in step 210 in order to establish a diagnosis of the individual that can be used in determining the provision of medication and other matters related to the measured levels. Specifically, sum level G can be used to assess whether the substances of each pair are present in a homeostatic relationship relative to one another (e.g. whether D and E are present in a homeostatic relationship to enable the production of an adequate level of RNA, specifically mRNA IL-1, to achieve or maintain a medically assessed healthy state). This in turn may be used to determine whether the addition or remediation of one of the substances in the pair is necessary to maintain or restore the homeostatic relationship, either by adding an amount of one or more of the substances or by adding an amount of one or more modulators.

Other methods for determining the relationship between the pair of substances, such as vector analysis and the calculation of the ratio between the levels of the substances in a pair, may also be used.

In one embodiment, the comparative standard levels of D, E, F and G may be a predetermined amount applicable to groups of individuals sharing one or more characteristics (e.g. age, sex, diseases, etc.). Alternatively, the comparative standard levels of D, E, F and G may be specific to an individual medically assessed as healthy.

An example of this is provided below using hypothetical numbers so as to make understanding of the process easier.

Measuring aluminum 22 via a blood test results in a number for D that results in a number 2, from within a range of 1-10. Measuring iron 24 via a blood test results in a number for E of 6 from within a range of 1-10. Measuring phosphorous 26 via a blood test results in a number for F of 6 from within a range of between 1-10. These numbers are then totaled and results in a G value of 14. A value for G between 20 and 30 may indicate a normal level.

The present invention provides the biological foundation that will enable the monitoring of relationships of modulated pairs substances in the body as they relate to medical care; including wellness, prevention and treatment pertaining to the mind and body.

The following are examples of the benefits of measuring the respective levels of minerals.

(1) Diets influenced by chemicals or substances processes with or influenced by minerals, foods with high levels of natural minerals, and herbal preparations that were grown in soil or water influences by minerals can be ineffective or cause serious consequences if dosage does not correspond properly to substances that are influenced through ions relationships. Creating a baseline assessment of an individual's levels of minerals can provide a foundation from which dosing can be tailored to meet specific requirements for maintenance or remediation of imbalances.

(2) Assessment of homeostatic imbalances of cellular absorption rates, and neural firing rates and the relationship between firing rates and absorption rates can enable therapies to be adjusted to compensate for the influences of absorption in the addition of substances to maintain or restore homeostasis.

(3) Rates of activity and absorption will dictate the rate of use or dissipation of nutrients essential to provide energy to maintain bodily processes and functions; including but not limited to the creation and maintenance of the body's defenses (i e immune system).

The present invention may potentially be related to a number of other pending applications by the inventor, such as U.S. patent application Ser. No. 13/013,216 filed on Jan. 25, 2011; U.S. patent application Ser. No. 12/701,076 filed on Feb. 5, 2010 and published as U.S. Patent Application Publication No. 2010/0204329; U.S. patent application Ser. No. 12/897,282 filed Oct. 4, 2010 and published as U.S. Patent Application Publication No. 2011/0020313; and U.S. patent application Ser. No. 12/701,083 filed on Sep. 21, 2010 and published as U.S. Patent Application Publication No. 2010/0203579. In particular, these applications may pertain to methods for measuring and analyzing the substances described in the present invention.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

What is claimed is:

1. A method for establishing a diagnosis based upon compared levels of substances:
measuring a level of nickel in an individual;
measuring a level of zinc in the individual;
measuring a level of boron in the individual;
comparing the measured levels;
establishing a diagnosis based upon the compared measured levels;
adding or subtracting an additional amount of nickel in the individual to ensure homeostasis based upon the compared measured levels and the established diagnosis;
adding or subtracting an additional amount of zinc in the individual to ensure homeostasis based upon the compared measured levels and the established diagnosis; and
adding or subtracting an additional amount of boron in the individual to ensure homeostasis based upon the compared measured levels and the established diagnosis.

2. A method for establishing a diagnosis based upon compared levels of substances:
measuring a level of calcium in the individual;
measuring a level of magnesium in the individual;
measuring a level of chloride in the individual;
comparing the measured levels;
establishing a diagnosis based upon the compared measured levels;
adding or subtracting an additional amount of the mineral calcium in the individual to ensure homeostasis based upon the compared measured levels and the established diagnosis;
adding or subtracting an additional amount of the mineral magnesium in the individual to ensure homeostasis based upon the compared measured levels and the established diagnosis; and
adding or subtracting an additional amount of the chloride in the individual to ensure homeostasis based upon the compared measured levels and the established diagnosis.

3. A method for establishing a diagnosis based upon compared levels of substances:
measuring a level of aluminum in the individual;
measuring a level of the iron in the individual;
measuring a level of the protein phosphorous in the individual;
comparing the measured levels;
establishing a diagnosis based upon the compared measured levels;
adding or subtracting an additional amount of aluminum in the individual based upon the compared measured levels and the established diagnosis;
adding or subtracting an additional amount of iron in the individual based upon the compared measured levels and the established diagnosis; and
adding or subtracting an additional amount of phosphorous in the individual based upon the compared measured levels and the established diagnosis.
